(19) United States
(12) Patent Application Publication
(10) Pub. No.: US 2014/0194507 A1
(43) Pub. Date: Jul. 10, 2014

(54) COMPOSITIONS FOR TREATMENT OF CANCER

(71) Applicant: NovaRx Corporation, San Diego, CA (US)

(72) Inventors: Feridoun KARIMI-BUSHERI, Edmonton (CA); Habib FAKHRAI, La Jolla, CA (US)

(21) Appl. No.: 13/801,920

(22) Filed: Mar. 13, 2013

Related U.S. Application Data
(60) Provisional application No. 61/749,214, filed on Jan. 4, 2013.

(51) Int. Cl. C07D 307/73

(52) U.S. Cl. CPC C07D 307/73
USPC 514/472; 549/482

(57) ABSTRACT

Compounds that are specifically toxic to cancer stem cells are disclosed.

### CROSS-REFERENCE TO RELATED APPLICATION

This application claims priority from U.S. provisional application 61/749,214 filed 4 Jan. 2013. The contents of this document are incorporated herein by reference.

### TECHNICAL FIELD

The invention is in the field of pharmaceuticals useful in cancer treatment. More particularly, it relates to small molecules that are particularly toxic to cancer stem cells.

### BACKGROUND ART

It has been recognized for some time that the cells contained in a particular cancer are heterogeneous and have different susceptibilities to chemotherapeutic treatments as well as radiation therapy. In particular, many cancers contain cancer stem cells that are chiefly responsible for the initiation and spread of the cancer, including metastases. Cancer stem cells are resistant to conventional therapy and it is particularly important to eradicate these cells to prevent progression and metastasis of cancer.

Karimi-Busheri, F., et al., J. Stem Cells (2012) 6:______ reported the isolation of stem cells both from non-small cell lung tumor specimens and from lung tumor cell lines and provide a system for identification of such stem cells using CD38 in combination with CD24 as biomarkers and a combination of these biomarkers with overexpression of ALDH1 and EpCAM. These markers provide a signature for tumor-initiating cells in the H-460 lung cancer cell line.

It has been reported by Bernstein, N. K., et al., Anticancer Agents in Med. Chem. (2008) 8:358-367 that a polynucleotide kinase which is essential for DNA repair is critical to the resistance of cancer cells, including cancer stem cells, to DNA-damaging agents. This paper suggests that inhibitors of polynucleotide kinase would be effective in overcoming the resistance of these cells to radiation therapy and to other forms of chemotherapeutic treatment.

PCT publication No. WO2010/139069 assigned to the University of Alberta discloses a series of 6,7a-dihydro-1H-pyrrolo[3,4-b]pyridine-5,7(2H,4aH) diones as inhibitors of this enzyme. This work was also reported by the same group in an article by Freschauf, G. K., et al., Cancer Res. (2009) 69:7739-7746.

There remains a need for additional small molecule pharmaceuticals that will provide effective treatment for cancer cells, including specifically cancer stem cells and that will overcome any resistance of these cells to additional chemotherapeutic agents.

### DISCLOSURE OF THE INVENTION

The present invention is related to pharmaceutical compositions that have been demonstrated to have enhanced ability to effect cell death and DNA destruction in cancer stem cells even in comparison to their capacity to do so in other cancer cells.

Accordingly, in one aspect, the invention is directed to pharmaceutical compositions comprising as an active ingredient a compound of formula (1)

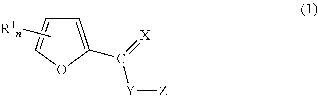

or a pharmaceutically acceptable salt thereof,

wherein each R¹ is a non-interfering substituent independently selected from the group consisting of halo, nitro, OR², OCOR², COOR², R²NCOR², CONR²₂, COR², NR²₂, S(O)ₘR², —CN and R³,

where each R² is independently H or alkyl (1-4)
R³ is alkyl (1-6) or substituted alkyl (1-6) wherein the substituents are independently halo, OR² or NR²₂,
m is 0, 1 or 2,
n is 0, 1 or 2,

X is O or S,

Y is NR or O,

Z is CR²A₂ or A,
wherein each A is independently phenyl optionally substituted by 1-3 R⁴ substituents wherein R⁴ is defined the same as R¹ as above.

In other aspects, the invention is directed to methods to treat cancers in subjects using the invention pharmaceutical compositions or active ingredients thereof.

### BRIEF DESCRIPTION OF THE DRAWINGS

FIG. 1 shows typical results of an assessment of the effect of the compounds of the invention on the viability of adherent cells vs. stem cells derived from the H460 cell line.

### MODES OF CARRYING OUT THE INVENTION

The pharmaceutical compositions of the invention contain as at least one active ingredient a compound of formula (1) as defined above. The alkyl groups in formula (1) may be straight or branched chain or cyclic and include, for example, methyl, ethyl, tertiary butyl, cyclopentyl, and the like. Methods for synthesizing the compounds of formula (1) are well known in the art and representative members of this genus are commercially available.

The “alkyl” groups in the compounds described herein may be straight or branched chain or cyclic and include, for example, methyl, ethyl, tertiary butyl, cyclopentyl, and groups that, in light of the ordinary skill in the art and the teachings provided herein, would be considered equivalent to any one of the foregoing examples. The alkyl group may have from 1 to 12 carbon atoms in the chain. Particular alkyl groups are those having 1 to 8 carbon atoms, 1 to 6 carbon atoms, or 1 to 4 carbon atoms.

The term “cycloalkyl” refers to a monocyclic, or fused, bridged, or spiro polycyclic ring structure that is saturated or partially saturated and has from 3 to 12 carbon atoms. Particular heterocycloalkyl groups are those having from 3 to 8 carbon atoms or from 5 to 7 carbon atoms. Illustrative examples of cycloalkyl groups include cyclopropane, cyclopropene, cyclobutane, cyclobutene, cyclopentane, cyclopentadiene, cyclohexane, cyclohexene, cyclohexadiene, cycloheptane, cyclooctane, adamantine, and groups that, in light of the ordinary skill in the art and the teachings provided herein, would be considered equivalent to any one of the foregoing examples.

Any formula given herein is intended to represent compounds having structures depicted by the structural formula as well as certain variations or forms. In particular, compounds of any formula given herein may have asymmetric centers and therefore exist in different stereoisomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds described herein include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds described herein and prodrugs thereof can generally be prepared by carrying out the procedures disclosed in the schemes or in the examples and preparations described below by substituting a readily available isotopically labeled reagent for a non-isotopically labeled reagent.

Pharmaceutically acceptable salts include salts of a free acid or base of a compound represented herein that is non-toxic, biologically tolerable, or otherwise biologically suitable for administration to the subject. See, generally, Berge, S. M., et al. “Pharmaceutical Salts,” J. Pharm. Sci. (1977) 66:1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

For treatment purposes, pharmaceutical compositions comprising compounds described herein may further comprise one or more pharmaceutically-acceptable excipients. A pharmaceutically-acceptable excipient is a substance that is non-toxic and otherwise biologically suitable for administration to a subject. Such excipients facilitate formulation and administration of a compound described herein and are compatible with the active ingredient. Examples of pharmaceutically-acceptable excipients include stabilizers, lubricants, surfactants, diluents, anti-oxidants, binders, coloring agents, emulsifiers, or taste-modifying agents. In preferred embodiments, pharmaceutical compositions are sterile compositions.

The pharmaceutical compositions described herein may be formulated as solutions, emulsions, suspensions, or dispersions in suitable pharmaceutical solvents or carriers, or as pills, tablets, lozenges, suppositories, powders for reconstitution, or capsules along with solid carriers according to conventional methods known in the art for preparation of various dosage forms. For topical applications, the compounds described herein are preferably formulated as creams or ointments or a similar vehicle suitable for topical administration.

For treatment of subjects using the pharmaceutical compositions of the invention or the compounds described herein per se, a variety of protocols and methods of administration may be employed depending on the nature of the subject, the particular kind of tumor, the judgment of the practitioner and the stage of cancer. Various methods of administration are known in the art including parenteral administration, oral or other digestive system-based administration, transmucosal, transdermal administration or administration by suppository. Parenteral administration may include IP, IV and subcutaneous forms.

The term “treat” or “treating” as used herein is intended to refer to administration of a compound described herein to a subject for the purpose of creating a therapeutic benefit. Treating includes reversing, ameliorating, alleviating, inhibiting the progress of, or lessening the severity of, a disease, disorder, or condition, or one or more symptoms of cancer. The term “subject” refers to a patient in need of such treatment. The subjects in important aspects of the invention are human subjects, but treatment is not limited to them. Treatment for malignancy is also important in other vertebrate species including various forms of livestock such as cows, pigs, sheep and goats as well as companion animals, fish and birds. In particular, the compounds described herein may be used to treat laboratory animal tumor models, such as rats, mice, rabbits and the like in order to optimize dosage regimens and protocols.

In treatment methods provided herein, “an effective amount” means an amount or dose sufficient to generally bring about the desired therapeutic benefit in subjects needing such treatment. Effective amounts or doses of the compounds described herein may be ascertained by routine methods, such as modeling, dose escalation or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about 1 ug to 2 mg of active compound per kilogram of subject's body weight per day, preferably about 0.05 to 100 mg/kg/day, or about 1 to 35 mg/kg/day, or about 0.1 to 10 mg/kg/day. The total dosage may be given in single or divided dosage units (e.g., BID, TID, QID).

As noted above, the compounds described herein are particularly effective in decreasing the viability of cancer stem cells in addition to their ability to effect cell death in non-stem cell forms of cancer—i.e., adherent cells. Accordingly, the pharmaceutical compositions of the invention or the compounds described herein per se are useful in treating subjects harboring malignant tumors.

The subjects in important aspects of the invention are human subjects, but treatment is not limited to them. Treatment for malignancy is also important in other vertebrate species including various forms of livestock such as cows, pigs, sheep and goats as well as companion animals, fish and birds. In particular, the compounds of formula (1) may be used to treat laboratory animal tumor models, such as rats, mice, rabbits and the like in order to optimize dosage regimens and protocols.

For treatment of subjects using the pharmaceutical compositions of the invention or the compounds of formula (1) per se, a variety of protocols and methods of administration may be employed depending on the nature of the subject, the particular kind of tumor, the judgment of the practitioner and the stage of cancer. Various methods of administration are known in the art including parenteral administration, oral or other digestive system-based administration, transmucosal, transdermal administration or administration by suppository. Parenteral administration may include IP, IV and subcutaneous forms.

The mode of administration will also depend on the nature of the formulation employed. Suitable formulations may be found in Remington's Pharmaceutical Sciences, latest edition, Mack Publishing Co., Easton, Pa. The compositions may be simple formulations with conventional excipients or may include liposomes, micelles, controlled release systems or other polymeric supports and may include other active ingredients unrelated to the compounds of formula (1). For solid tumors, it is also possible to provide the compounds of formula (1) directly by intratumoral administration. Dosage levels are variable depending on the practitioner's judgment and the nature of the subject but can readily be determined from the behavior of the compounds in animal models commonly used to optimize dosages.

Various specific embodiments of the compound of formula (1) including its salts include: X═O and Y═O or X═O and Y═NR for instance where N is H or X═S and Y═NR for instance where N is H wherein, in each said case, Z is CR²A₂ or wherein in each said case Z is A, or embodiments in each of these cases where R¹ is selected from any 2 of the substituents listed therefor, or embodiments in each of these cases where n is 0. In each of these cases, each A may be unsubstituted phenyl. For each of the embodiments mentioned, R¹ as a substituent on the furan may be halo or nitro when n is 1 or 2. For each of the embodiments mentioned, the substituents on A may be alkoxy or alkyl, for instance, methoxy or methyl.

As used herein, “a” or “an” means one or more than one unless otherwise indicated or clear from the context. All documents cited are incorporated herein by reference.

The following examples are intended to illustrate but not to limit the invention.

### EXAMPLE 1

### Isolation of Stem Cells

Stem cells were isolated from non-small cell lung tumor specimens of human tumors developed in NOD/SCID mice according to the description set forth in Karimi-Busheri, F., et al., J. Stem Cells (2012) supra, incorporated herein by reference. Briefly, a combination of three criteria were used:

Formation of a subpopulation of cells identifiable by efflux of Hoechst 33342 by ABC transporters via flow cytometry analysis;

Formation of floating spheres in culture; and

Expression of the markers CD133, and CD24/CD38 ratio. These enriched populations of stem cells were used in the experiments below.

EXAMPLE 2

### Viability Assay

The compounds of formula (1) showed activity in a cell viability assay conducted as follows:

Samples of 5×10³ cells per well were seeded onto 96-well plates along with the desired concentration of compound of formula (1) dissolved in DMSO. Control wells contained cells without treatment and medium without cells was used as a background control. The plates were incubated for 120 hours at 37° C. and then assayed for viability using the commercially available PrestoBlue® Cell Viability Staining assay marketed by Invitrogen, Carlsbad, Calif. After incubation, the plates were irradiated at 540-570 nm and the emission at 590 nm measured. Cell death was shown by a decrease in fluorescence.

The compounds of formula (1) are able to decrease viability by at least 40% in this assay when the concentration of compound is 100 μM.

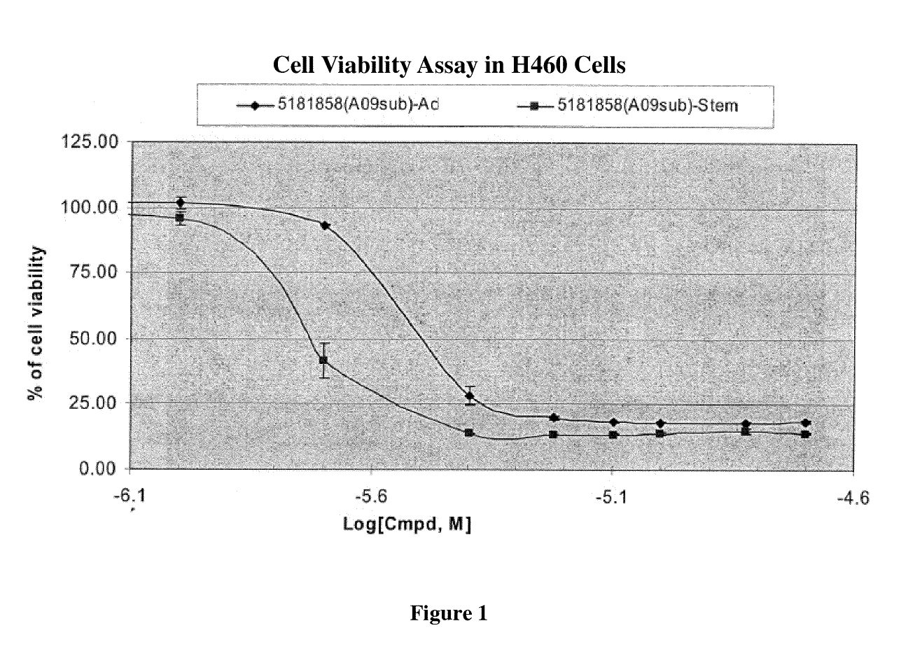

Varying concentrations of the compounds of formula (1) were used to determine IC₅₀. The compounds of formula (1) have IC₅₀'s between 1 and 5 μM. A typical result is shown in FIG. 1. As shown, the efficacy of the compound is greater on stem cells than in adherent cancer cells.

### EXAMPLE 3

### Determination of Maximum Tolerated Dose

For each compound tested in this assay, 10 female BALB/c nu/nu mice were used in an in vivo study to determine maximum tolerated dose (MTD). An MTD of 25 μM was found for these compounds.

### EXAMPLE 4

### In Vivo Efficacy Model

NOD/SCID mice containing human tumor stem cells of a non-small cell lung tumor specimen are used to evaluate dosage levels for the compounds of formula (1).

### EXAMPLE 5

### Exemplary Compounds

The following compounds are typical of those of formula (1):

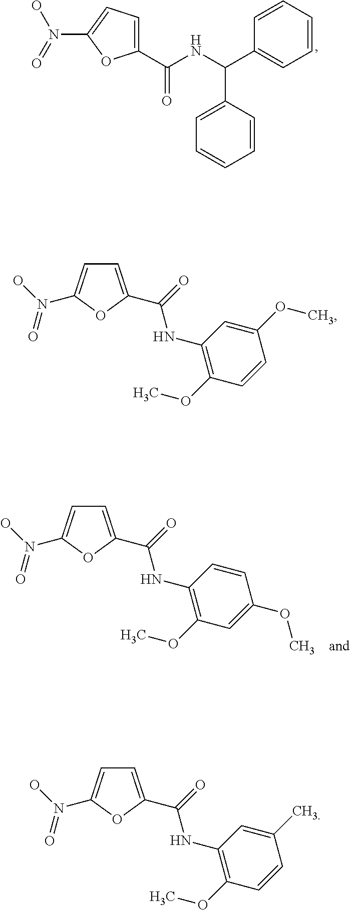

1. A pharmaceutical composition comprising as an active ingredient a compound of formula (1) 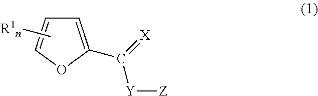
wherein each R¹ is a non-interfering substituent independently selected from the group consisting of halo, nitro, OR², OCOR², COOR², R²NCOR², CONR²₂, COR², NR²₂, S(O)ₘR², —CN and R³,
where each R² is independently H or alkyl (1-4)
R³ is alkyl (1-6) or substituted alkyl (1-6) wherein the substituents are halo, OR² or NR²₂,
m is 0, 1 or 2,
n is 0, 1 or 2,
X is O or S,
Y is NR or O,
Z is CR²A₂ or A,
wherein each A is independently phenyl optionally substituted by 1-3 R⁴ substituents wherein R⁴ is defined the same as R¹ as above,
or the pharmaceutically acceptable salts thereof.

2. The composition of claim 1 wherein
X is O and Y is NR.

3. The composition of claim 1 wherein
R¹ is halo or NO₂.

4. The composition of claim 1 wherein each
R⁴ is independently OR² or R².

5. The composition of claim 1 wherein formula (1) is

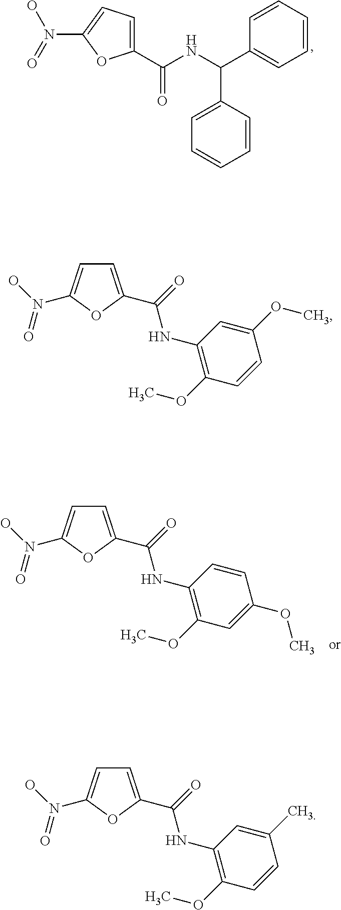

6. A method to treat cancer in a subject in need thereof which comprises administering to said subject an effective amount of the composition of claim 1 or at least one compound of formula (1) or its salt.
